(19) United States
(12) Patent Application Publication
(10) Pub. No.: US 2006/0193921 A1
(43) Pub. Date: Aug. 31, 2006

(54) Hydrophilic beads for use in topical formulations

(76) Inventors: J. Steve Brown, Gilbert, AZ (US); John Hill, Mesa, AZ (US); J. Randall Tryon, Mesa, AZ (US); Michele Ward, Queen Creek, AZ (US)

Correspondence Address: NOBLITT & GILMORE, LLC., 4800 NORTH SCOTTSDALE ROAD, SUITE 6000, SCOTTSDALE, AZ, 85251, US

(21) Appl. No.: 11/340,310

(22) Filed: Jan. 26, 2006

Related U.S. Application Data
(60) Provisional application No. 60/647,530, filed on Jan. 26, 2005.

(51) Int. Cl. A61K 36/886; A61K 8/73; A61K 9/14

(52) U.S. Cl. 424/489; 424/70.13; 424/744; 424/63

(57) ABSTRACT

A composition and method for producing hydrophilic beads for use in topical formulations is described as generally including gellan gum, salts of divalent cations and water. Disclosed features and specifications may be variously controlled, adapted or otherwise optionally modified to realize improved production and/or use of hydrophilic beads to achieve a particular desired purpose. Exemplary embodiments of the present invention generally provide hydrophilic beads that may be comixed with cosmetic, cosmeceutical and/or topical preparations for surface abrasion or as carriers for the delivery of other compounds; such as, aloe vera, ascorbic acid, active agents, and/or the like.

### RELATED APPLICATIONS

This application claims the benefit of U.S. Provisional Patent Application Serial No. 60/647,530 filed in the United States Patent and Trademark Office on Jan. 26, 2005 by Steve Brown, John Hill, Randy Tryon, and Michele Ward.

### FIELD OF INVENTION

The present invention generally relates to topical preparations; and more particularly, representative and exemplary embodiments of the present invention generally concern hydrophilic beads for use with topical formulations.

### BACKGROUND OF INVENTION

Micro-encapsulation is a process in which relatively small particles or droplets are surrounded by a coating to produce capsules with many useful properties. In its simplest form, a microcapsule is a small sphere with a substantially uniform wall at its periphery. The material inside the microcapsule is generally referred to as the core, internal phase or fill, whereas the wall is sometimes called a shell, coating or membrane. Most microcapsules have diameters between a few micrometers and a few millimeters.

Many microcapsules, however, bear little resemblance to simple spheres. The core may be a crystal, a jagged adsorbent particle, an emulsion, a suspension of solids, or a suspension of smaller microcapsules. The microcapsule may even have multiple walls.

The reasons for employing microencapsulation are at least as varied as the processes which may be used in their manufacture. In some cases, the core must be substantially isolated from its surroundings, as in segregating materials from the deteriorating effects of oxygen, retarding evaporation of a volatile core, improving the handling properties of a sticky material, or isolating a reactive core from chemical exposure. In other cases, the objective may not be to isolate the core completely, but to control the rate at which the core material leaves the microcapsule, as in the controlled release of drugs or pesticides. Representative applications may be as simple as masking the taste or odor of the core, or as complex as increasing the selectivity of an adsorption or extraction process.

The pan coating process, widely used in the pharmaceutical industry, is among the oldest industrial procedures for forming small, coated particles or tablets. The particles are tumbled in a pan or other device while the coating material is applied slowly.

Air-suspension coating of particles by solutions or melts provides better control and flexibility. The particles are coated while suspended in an upward-moving air stream. They are supported by a perforated plate having different patterns of holes inside and outside a cylindrical insert. Just a sufficient quantity of air is permitted to rise through the outer annular space to fluidize the settling particles. Most of the rising air (typically heated) flows inside the cylinder, causing the particles to rise relatively rapidly. At the top, as the air stream diverges and slows, the particles settle back onto the outer bed and move downward to repeat the cycle. The particles may pass through the inner cylinder many times in a just a few minutes.

Liquids may be encapsulated using a rotating extrusion head containing concentric nozzles. In this process, a jet of core liquid is surrounded by a sheath of wall solution or melt. As the jet moves through the air it breaks (due to Rayleigh instability) into droplets of core, each coated with the wall solution. While the droplets are in flight, a molten wall may be hardened or a solvent may be evaporated from the wall solution. Since most of the droplets are within ±10% of the mean diameter, they land in a relatively narrow ring around the spray nozzle. Hence, if needed, the capsules may be hardened after formation by catching them in a ring-shaped hardening bath. This process is well-suited for forming particles on the order of 400-2000 μm in diameter. Since the drops are produced by the breakup of a liquid jet, the process is generally only suitable for liquid or slurry preparations. This process generally offers a relatively high production (i.e., up to about 22.5 kg of microcapsules per nozzle per hour per head).

Spray drying serves as a microencapsulation technique when an active material is dissolved or suspended in a melt or polymer solution and becomes trapped in the dried particle. The main advantage of spray drying is the ability to handle labile materials, due to the short contact time in the dryer. Additionally, the operation is economical. In modern spray dryers the viscosity of the solutions to be sprayed may be as high as 300 mPa·s.

With interfacial polymerization microencapsulation, two reactants in a polycondensation meet at an interface and react relatively quickly. The basis of this method is the classical Schotten Baumann reaction between an acid chloride and a compound containing an active hydrogen atom (e.g., proton donor), such as an amine or alcohol, polyesters, polyurea, polyurethane, etc. Under the right conditions, thin flexible walls form rapidly at the interface. For example, a solution of a pesticide and a di-acid chloride may be emulsified in water with an aqueous solution containing an amine and a polyfunctional isocyanate added. Base is generally present to neutralize the acid formed during the reaction with condensed polymer walls formed at the interface of the emulsion droplets.

In a few microencapsulation processes, the direct polymerization of a single monomer may be achieved on the particle surface. In one representative process, cellulose fibers may be encapsulated in polyethylene while immersed in dry toluene. Typical deposition rates are on the order of about 0.5 μm/min with coating thickness ranging between 0.2-75 μm. The coating is generally uniform, even over sharp topological morphologies and projections.

In a number of other processes, a core material may be imbedded in a polymeric matrix during formation of the particles. A simple method of this type is that of spray-drying, in which the particle may be formed by evaporation of the solvent from the matrix material. However, the solidification of the matrix may also be caused by a chemical modification.

Even when the aim of a microencapsulation technique is the isolation of the core from its surrounding, the wall must generally be ruptured at the time of use. Many walls may be ruptured relatively easily by pressure or shear stress, as in the case of the breakage of dye particles during writing on a copy sheet to form a copy. Capsule contents may be released by melting the wall, or dissolving under particular conditions, as in the case of an enteric drug coating. In other applications, the wall may be broken by solvent action, enzyme digestion, chemical reaction, hydrolysis or slow disintegration.

Microencapsulation may also be used to slow the release of a drug into the body. This may permit a single controlled release dose to substitute for several doses of non-encapsulated drug, and may also decrease toxic side effects for some drugs by preventing high initial concentrations in the blood. There is usually a certain desired release protocol. In some cases, it is zero-order, i.e. the release rate is constant. In that case, the microcapsules deliver a fixed amount of drug per minute or hour during the period of their effectiveness. This may occur as long as a solid reservoir or dissolving drug is maintained inside the microcapsule.

A more typical release pattern is first-order, in which the delivery rate decreases exponentially with time until the drug material is depleted. In this situation, a fixed amount of drug may be disposed in solution inside the microcapsule. The concentration ratio between the inside and the outside of the capsule generally decreases continually as the drug diffuses.

Given the varied techniques that have been used for the manufacture of microparticles in the past, conventional methods for producing water-based microspheres are generally based on agar or gelatin with ‘shell-and-core’ construction for maintaining particle geometries.

### SUMMARY OF THE INVENTION

In representative aspects, the present invention provides compositions and methods for providing substantially homogenous gellan-based hydrophilic microsphere beads for use in topical formulations. Advantages of the present invention will be set forth in the Detailed Description which follows and may be apparent from the Detailed Description or may be learned by practice of exemplary embodiments of the invention. Still other advantages of the invention may be realized by means of any of the instrumentalities, methods or combinations particularly pointed out in the claims.

### DETAILED DESCRIPTION OF EXEMPLARY EMBODIMENTS

The following representative descriptions of the present invention generally relate to exemplary embodiments and the inventors' conception of the best mode, and are not intended to limit the applicability or configuration of the invention in any way. Rather, the following description is intended to provide convenient illustrations for implementing various embodiments of the invention. As will become apparent, changes may be made in the function and/or arrangement of any of the elements described in the disclosed exemplary embodiments without departing from the spirit and scope of the invention.

Various representative implementations of the present invention may be applied to any system for providing a hydrophilic particle for use in topical formulations. As used herein, the terms “bead”, “particle”, “sphere”, or any variation or combination thereof, are generally intended to include anything that may be regarded as at least being susceptible to characterization as, or generally referring to a discrete formulation component taken either alone or in combination with a carrier solution.

A detailed description of an exemplary application, namely a composition and method for delivering a topical application of aloe vera via a gellan-based hydrophilic particle, is provided as a specific enabling disclosure that may be generalized to any application of the disclosed composition and method for delivering any type of active ingredients via hydrophilic beads in accordance with various embodiments of the present invention.

A representative hydrophilic bead formulation is disclosed as comprising water, gellan gum and at least one salt of a divalent cation. Additional formulation components may include various traffic compounds comprising, for example, preservatives, coloring agents, fragrances, active ingredients (aloe, ascorbic acid, etc.) and/or the like.

An exemplary bead composition generally provides substantially spherical beads having substantially uniform diameters in the range of up to about 50 microns to approximately more than 5,000 microns. In an exemplary application, in accordance with a representative embodiment of the present invention, the beads may be visible.

In general, the disclosed beads may be suitably adapted to be ‘soft to the touch’ and at least partially disintegrate when rubbed, for example, against the skin. Suitably adapted beads generally provide a substantially homogenous matrix of material (i.e., the beads generally have no shell or other non-homogenous components for maintaining the particle shape or size). In certain exemplary embodiments of the present invention, the disclosed hydrophilic beads are typically opaque, fully transparent, partially transparent or otherwise translucent. In general, the beads may be characterized as hydrophilic particles comprising gellan gum that are suitably adapted to optionally carry or otherwise deliver traffic compounds, such as, for example:

fruit and vegetable extracts and juices, including, for example: apple, apricot, asparagus, beet, black currant, blackberries, boysenberry, broccoli, cabbage, carrot, celery, cherry, cranberry, red currant, elderberry, garlic, gooseberry, grape, grapefruit, lemon, lettuce, lime, loganberry, mustard, onion, orange, parsley, passion fruit, pea, peach, pear, pineapple, plum, prune, quince, raspberry, rhubarb, spinach, squash, strawberry, tangerine, tomato, turnip, watercress, and/or the like;

alpha and beta hydroxy acids, such as, for example: ascorbic acid, glycolic acid, lactic acid, malic acid, oxalic acid, salicylic acid, tartaric acid, and/or the like;

hyaluronic acid;

natural antibiotics, such as, for example: mycosubtilin, actidione, nisin, pimaricin, microsubtilin, patuline, and/or the like;

vitamins, such as, for example: vitamin C, vitamin B₁ (thiamin), vitamin B₂ (riboflavin), niacin (nicotinic acid, nicotinamide, vitamin PP), vitamin H (biotin), vitamin B₆, vitamin B₁₂, and/or the like;

amino acids, such as, for example: alanine, valine, leucine, tyrosine, glutamic acid, tryptophan, methionine, lysine, iosleucine, phenylalanine, glycine, cystine, aspartic acid, histidine, arginine, ornithine, serine, asparagines, praline, aminobutyric acid, threonine, and/or the like;

a protein, a peptide, a peptide combination, and/or the like;

polar extracts of fragrance materials, such as, for example: agrumen oil, allium oil, ambergris, ambrette seed, amyris oil, angelica root, angelica seed, anise, aniseed oil, Artemisia oil, balm mint, balsam fir oil, basil oil, bay oil, bergamot oil, birch tar oil, bitter almond oil, bois de rose oil, buchu leaf oil, cabreuva oil, calamus oil, camphor, cananga oil, caraway oil, cardamom oil, carrot seed oil, cassia oil, castoreum, cedar leaf oil, cedar oil, celery seed oil, chamomile oil, cinnamon bark oil, cistus oil, citronella oil, civet, clary sage oil, clove oil, cognac oil, cola, copaiba oil, coriander oil, cornmint oil, costus root oil, cumin oil, cypress oil, dill seed oil, dill weed oil, elemi oil, estragon oil, eucalyptus oil, eugenol, fennel oil, fir needle oil, galbanum oil, garlic oil, geranium oil, ginger grass oil, ginger oil, grapefruit oil, green cognac oil, guiaiac wood oil, gurjun balsam oil, helichrysum oil, hemlock oil, hiba oil, jasmine oil, juniper berry oil, labdanum oil, laurel leaf oil, lavandin oil, lavandula oil, lavender oil, leek oil, lemon grass oil, lime oil, linaloe oil, litsea cubeba oil, lovage oil, mandarin oil, marjoram oil, menthe arvensis oil, menthe piperita oil, musk, musk ambrette, myrrh oil, nerol, neroli oil, nutmeg oil, oakmoss oil, olibanum oil, onion oil, opopanax oil, orange oil, origanum oil, orris root oil, palmarosa oil, parsley herb oil, patchouli oil, pennyroyal oil, rose oil, rosewood oil, rue oil, sage oil, sandalwood oil, sassafras oil, savory oil silver fir oil, spearmint oil, spike lavender oil, spruce oil, star anise oil, tarragon oil, tea tree oil, terpineols, thujopsene, thyme oil, tolu balsam oil, valerian oil, vanilla oil, verbena oil, vetiver oil, violet leaf alcohol, wood turpentine oil, ylang-ylang oil, and/or the like;

polar extracts or juices of botanicals, such as, for example: acacia, abies alba, abietin, absinth, acacia decurrens (mimosa), acacia excelsa (bois de rose), aleppy cardamom, allsprice, aloe vera (aloe barbadensis), aloe perei, algae, anhydrol mate, anhydrol tea, apiole (parsley), apium graveolens (celery seed), arrowroot, armoracia lapathifolia (horseradish), arnica, asarum canadense (snakeroot), ascophyllum, aspergillus oryzae (soybean), azulene (chamomile), banana, bamboo, bay laurel, betula alba (birch bud), birch, birch leaf, blazing star (deertongue), black walnut, bladderwrack, boronia, boswellia (olibanum), bruyere, buchu, burdock, butterfly lily, cade, cajuput, calamus, calendula officialis (marigold), capsicum, clove, coltsfoot, comfrey, coneflower, cedar, coconut, coffee, cactus, cedar, chapparal, chickweed, cedarwood, cherry birch, cicely (anise), cistus, citrus aurantium (neroli, orange flower, petitgrain), cucumber, cyclotene (fluove, lovage, maple), cytisus scoparius (broom absolute), dacrydium elatum, dalbergia latifolia (bois de rose), dandelion, daucus (carrot seed), diorissimo (lily of the valley), eagle wood (agar), eau de brouts (orange flower, petitgrain water absolute), elder, elder flower, elemi, estragon, eucalyptus, eucaria spicata (sandalwood), eudesmol (araucaria concrete), evernia furfuracea (oakmoss, treemoss), exaltolide (musk), eyebright, farnesol (ambrette absolute), fennel, fenugreek, fern, ficus carica, firmoss, flouve ordorante, gardenia, gensing, geranium, ginger, glycyrrhiza glabra (licorice), goldenrod, goldenseal, gorse, guaiacwood, guava, garlic, grape, green tea, hayata (machilus), hemlock spruce, henna, hibiscus abelmoschus (ambrette seed), honey, hop absolute, horse chestnut, horsetail, hyacinth orientalis, hyssop, inchigrass, inula helenium (elecampane), ivy leaf, irish moss, jaborandi, jacmal (curacao peel), jasmine, jonquil, juniper, karma, keora (pandanus), kava-kava, lavender, lemon, lemon bioflavanoids, lemongrass, laminaria, lilac, lime, locus bean, lovage, machilus (hayata), magnolia, majorana hortensis (marjoram), macadamia, mallow, melon, mandarin oil, maple, mastic, mate, matricaria chamomilla (chamomile), mango, may-chang (litsea cubeba), melaleuca, Melissa officianalis, menthe citrate, mistletoe, moschus moschiferus, mugwort, myrrh, myrtle, narcissus jonquilla, nepeta cataria, nettle, nutmeg, oakmoss, oakbark, ocimum gratissimum, olive, opopanax, Oregon fir, origanum hirtum, orris paprika, passionflower, passionfruit, pellitory root, pennyroyal, perilla frutescens, petitgrain cedrat, phellandrene (elemi, angelica, eucalyptus), picea excelsa, pimenta berry, pine, pinetree, piper crassipes, piper longum, pollen, papaya, prunus armeniaca, pulegone (pennyroyal-moroccan), quinine, red clover, roman chamomile, rondeletia (bay leaf, clove bud, lavandin), rosa centifolia, rosa damascene, rose hips, rosemary, rose otto, rhatany, rice, rosewood, rosin, ruta graveolens (rue), saffron, sage clary, St. John's bread (carob), St. John's wort (everlasting absolute), salvia lavandulaefolia (rosemary), salvia hispanica (chia), salvia sclarea (sage clary), sandarac, santalum citrinum, schinus molle, smilax (sasparilla), sesame, sea buckthorn, sea fennel, sea kelp, spirulina, sesame, soap bark, sumbul root, sweet myrtle (calamus), syringe vulgaris (lilac), tagetes glandulifera, tagetes patula, tea tree (melaluca), thea sinensis, thyme, thymus capitatus, tolu balsam, toona calantas, tsuga Canadensis, tuberose, tumeric, ulex europaeus, valerian officinalis, vanilla, verbena, vetiver zizanoides, violet flower, viscum album (mistletoe), wallflower, walnut, wattle, wild cherry, witch hazel, wormseed, wormwood, xanthoxylum alatum, yarrow, ylang-ylang, yucca, zedoaria, zingerone officinale (ginger), and or the like; and

glitter, special effects pigments, color shifting pigments, and/or the like.

In an exemplary bead formulation, 0.1% to 5% of KELCOGEL AFT (gellan) was added to 10 mmolar CaCl₂ (in deionized water) at elevated temperature (e.g., 80 degrees Celsius) with 80 g GERMAZIDE M (preservative) and 20 g of a 0.1% solution of red dye no. 33 to produce approximately a 4 liter solution. Pink beads where formed with excellent production yield and virtually no waste. Collection of beads was easily achieved by scooping into pails. The beads where observed to be highly transparent with uniform spherical geometries. The beads were also observed to have been formed substantially irreversibly, which is to say that they could not be melted or otherwise returned to a liquid or semi-liquid state to again form beads. The beads were small to medium size and demonstrated mechanical disintegration with little to no residue remaining upon manual abrasion.

In yet another exemplary bead formulation, 0.1% to 5% of KELCOGEL AFT (gellan) was added to 10 mmolar CaCl₂ (in deionized water) at elevated temperature (e.g., 80 degrees Celsius) with 20 g of GERMAZIDE M. Approximately 0.5% of the formulation comprised a 0.1% solution of blue dye no. 1. In this formulation, larger beads were produced which were harder. These beads also demonstrated a brilliant clear blue color.

Hydrophilic beads, in accordance with various representative embodiments of the present invention, may be included in such topical formulations as, for example: shampoos; conditioners; gels; lotions; surfactant systems; hand sanitizer formulations; cosmetics; cosmeceutical formulations; toiletry formulations; and/or the like. Additional uses may include, for example, the incorporation of active ingredients: to provide a desired aesthetic appearance; to provide a desired product texture; to provide an abrasive and/or delivery vehicle for various topical formulations; to provide a transparent or translucent gel; to color or otherwise decorate a gel prior to purchase or use; and/or the like.

In a preferred embodiment, the hydrophilic beads may be suspended in a gel. As the consumer uses the gel, hydrophilic beads are rubbed against the skin or hair and abrade, leaving behind, for example, the active ingredient—but generally no substantial debris from the disintegration of the hydrophilic bead itself. Additionally, gellan gum generally provides the advantage of imparting substantially no objectionable skinfeel after use.

In another exemplary embodiment, gellan beads may be suitable adapted to provide a substantially mono-sized geometry on the order of about 1000 microns. They may be generally colored, at least semi-transparent and carry an active ingredient, such as, for example, aloe vera. The beads may be at least partially suspended in a substantially clear cosmetic product, where the beads are generally visible in order to improve the visual or aesthetic appeal of the product. The visual appeal of discrete beads suspended in a product formulation may further provide the consumer visual confirmation that an active ingredient is indeed present in the cosmetic or toiletry product.

It will be appreciated that gellan is a water-based gum that may be generally activated (e.g., thickened) by salts, especially salts of divalent cations, such as calcium, magnesium, and/or the like. It will be further appreciated that production of uniform bead geometry (i.e., mono-sizing) may be commercially desirable, but should not be construed to constitute a critical, required, or otherwise essential feature of the present invention. Similarly, coloring may also be commercially desirable, but should not be considered as essential or otherwise limiting. For example, a hydrophilic bead formulation comprising coloring in a cosmetic application may be desirable to enhance aesthetic appeal. In another exemplary embodiment, hydrophilic bead formulations containing coloring may provide proof of application once topically applied. In general, an un-pigmented bead would be colorless (i.e., “water white”) and appear as a bubble or jellyfish when suspended in solution. This aesthetic effect may also be commercially desirable in some applications of the present invention, but should not be construed as critical or essential.

The inclusion of active ingredients may also be commercially desirable or otherwise preferred, but not essential to the present invention. Active ingredients may be incorporated into, and carried by, the beads, thus demonstrating the utility of the invention in an exemplary and representative aspect. Active ingredients may include aloe vera, ascorbic acid (vitamin C), water-soluble vitamins, herbal extracts and infusions, fragrances, skin whitening agents, dyes, enzymes, insect repellants, salts, topical treatments, pharmaceutical preparations, and/or the like. The present invention anticipates that the disclosed bead formulations are well-suited for hydrophilic active ingredients; however, lipophilic active ingredients may also be incorporated using a variety of conventional chemical techniques.

Various exemplary and representative embodiments of the present invention may be adapted or otherwise suitable configured to provide topical formulators with a hydrophilic particle which serves several purposes. The particle may be customized to decorate a cosmetic product by being at least partially transparent; however, the disclosed particles may also be chemically or mechanically modified to provide a translucent or opaque effect as needed. The disclosed particles generally leave behind no substantial debris upon mechanical abrasion, thereby requiring no rinsing after application and use. The skinfeel of the gellan particles after use has been demonstrated as excellent, without gumminess or greasiness.

In the foregoing specification, the invention has been described with reference to specific exemplary embodiments; however, it will be appreciated that various modifications and changes may be made without departing from the scope of the present invention as set forth herein. The specification is to be regarded in an illustrative manner, rather than a restrictive one and all such modifications are intended to be included within the scope of the present invention. Accordingly, the scope of the invention should be determined by the exemplary embodiments and their legal equivalents rather than by merely the examples described above.

For example, the steps recited in any method or process embodiment may be executed in any order and are not limited to the specific order presented in the exemplary embodiments. Additionally, the components and/or elements recited in any apparatus or composition embodiment may be assembled or otherwise operationally configured in a variety of permutations to produce substantially the same result as the present invention and are accordingly not limited to the specific configuration recited in the exemplary embodiments.

Benefits, other advantages and solutions to problems have been described above with regard to particular embodiments; however, any benefit, advantage, solution to problem or any element that may cause any particular benefit, advantage or solution to occur or to become more pronounced are not to be construed as critical, required or essential features or components of the invention.

As used herein, the terms “comprising”, “having”, “including” or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

We claim:

1. A hydrophilic bead composition, comprising:
water;
gellan gum; and
at least one salt of a divalent cation.

2. The composition of claim 1, wherein said divalent cation comprises at least one of calcium chloride, magnesium and calcium.

3. The composition of claim 1, wherein said composition is suitably adapted to at least one of carry and deliver at least one traffic compound.

4. The composition of claim 3, wherein said traffic compound comprises at least one of: a preservative; a fruit extract; a fruit juice; a vegetable extract; a vegetable juice; an alpha hydroxy acid; a beta hydroxy acid; hyaluronic acid; an antibiotic; a natural antibiotic; a vitamin; an amino acid; a protein; a peptide; a peptide combination; a fragrance; a polar extract of fragrance material; a botanical material; a polar extract of a botanical material; a juice of a botanical material; glitter; a special-effect pigment; a coloring agent; a dye; a color shifting pigment; and an active agent.

5. The composition of claim 4, wherein said active agent comprises at least one of aloe and ascorbic acid.

6. The composition of claim 1, wherein said composition is substantially comixed in at least one of a cosmetic formulation, a cosmeceutical formulation and a topical formulation.

7. The composition of claim 1, further comprising at least one of an active ingredient, a hydrophilic active ingredient and a lipophilic active ingredient.

8. The composition of claim 7, wherein said active ingredient comprises at least one of aloe vera, ascorbic acid, a water-soluble vitamin, an herbal extract, an herbal infusion, a fragrance, a skin whitening agent, a dye, an enzyme, an insect repellant, a salt, a topical treatment and a pharmaceutical preparation.

9. The composition of claim 1, wherein said composition comprises a delivery vehicle for at least one of a cosmetic, a toiletry, and a topical preparation.

10. The composition of claim 1, wherein said composition comprises a plurality of hydrophilic beads having a substantially uniform diameter on the order of up to about 50 microns to approximately more than about 5,000 microns.

11. The composition of claim 1, wherein said composition is suitably adapted to at least one of:
feel substantially soft upon topical application;
demonstrate manipulability with respect to optical properties corresponding to at least one of transparence, translucence and opacity;
form a substantially homogenous matrix of bead material;
at least partially disintegrate upon manual abrasion; and
leave substantially no residue upon topical application.

12. A method for producing hydrophilic beads, said method comprising the steps of:
providing water;
providing gellan gum;
providing a salt of a divalent cation;
mixing said water and said divalent cation; and
introducing said gellan gum to said water and divalent cation mixture.

13. The method of claim 12, wherein said divalent cation is at least one of calcium chloride, magnesium and calcium.

14. The method of claim 12, wherein said composition is suitably adapted to at least one of carry and deliver at least one traffic compound.

15. The method of claim 12, wherein said traffic compound comprises at least one of: a preservative; a fruit extract; a fruit juice; a vegetable extract; a vegetable juice; an alpha hydroxy acid; a beta hydroxy acid; hyaluronic acid; an antibiotic; a natural antibiotic; a vitamin; an amino acid; a protein; a peptide; a peptide combination; a fragrance; a polar extract of fragrance material; a botanical material; a polar extract of a botanical material; a juice of a botanical material; glitter; a special-effect pigment; a coloring agent; a dye; a color shifting pigment; and an active agent.

16. The method of claim 12, wherein said active agent comprises at least one of aloe and ascorbic acid.

17. The method of claim 12, wherein said composition is substantially comixed with at least one of a cosmetic formulation, a cosmeceutical formulation and a topical formulation.

18. The method of claim 12, further comprising the step of providing at least one of an active ingredient, a hydrophilic active ingredient, and a lipophilic active ingredient.

19. The method of claim 18, wherein said active ingredient comprises at least one of aloe vera, ascorbic acid, a water-soluble vitamin, an herbal extract, an herbal infusion, a fragrance, a skin whitening agent, a dye, an enzyme, an insect repellant, a salt, a topical treatment and a pharmaceutical preparation.

20. The method of claim 12, wherein the resulting hydrophilic bead formulation comprises a delivery vehicle for at least one of a cosmetic, a toiletry, and a topical preparation.

21. The method of claim 12, wherein the resulting hydrophilic bead formulation comprises a plurality of substantially spherical beads having a substantially uniform diameter in the range on the order of up to about 50 microns to approximately more than 5,000 microns.

22. The method of claim 12, wherein the resulting hydrophilic bead formulation is suitably adapted to at least one of:
feel substantially soft upon topical application;
demonstrate manipulability with respect to optical properties corresponding to at least one of transparence, translucence and opacity;
form a substantially homogenous matrix of bead material;
at least partially disintegrate upon manual abrasion; and
leave substantially no residue upon topical application.

23. A method of formulating hydrophilic beads, said method comprising the steps of:
providing approximately 96% (wt/wt) of 10 mmolar calcium chloride in deionized water;
providing approximately 2% (wt/wt) of gellan gum;
providing approximately 2% (wt/wt) of preservative; and
introducing said gellan gum and preservative to said calcium chloride solution.

24. The method of claim 23, wherein said preservative comprises GERMAZIDE M.

25. The method of claim 23, optionally comprising the step of introducing approximately 0.5% (wt/wt) of 0.1% red dye no. 33.

26. The method of claim 24, wherein the resulting product comprises at least one of pink, transparent, translucent, opaque and substantially uniformly spherical beads.

27. The method of claim 23, wherein the resulting product is suitably adapted to at least one of:
feel substantially soft upon topical application;
demonstrate manipulability with respect to optical properties corresponding to at least one of transparence, translucence and opacity;
form a substantially homogenous matrix of bead material;
at least partially disintegrate upon manual abrasion; and
leave substantially no residue upon topical application.

28. A method of formulating hydrophilic beads, said method comprising the steps of:
providing approximately 95% (wt/wt) of 10 mmolar calcium chloride in deionized water;
providing approximately 3% (wt/wt) of gellan gum;
providing approximately 2% (wt/wt) of preservative; and
introducing said gellan gum and preservative to said calcium chloride solution.

29. The method of claim 28, wherein said preservative comprises GERMAZIDE M.

30. The method of claim 28, optionally comprising the step of introducing approximately 0.5% (wt/wt) of 0.1% blue dye no. 1.

31. The method of claim 30, wherein the resulting product comprises at least one of blue, transparent, translucent, opaque and substantially uniformly spherical beads.

32. The method of claim 28, wherein the resulting product is suitably adapted to form a substantially homogenous matrix of bead material.
